(19) United States
(12) Patent Application Publication
(10) Pub. No.: US 2024/0428302 A1
(43) Pub. Date: Dec. 26, 2024

(54) ESTIMATE ACCURACY SCORING MODEL

(71) Applicant: Experian Health, Inc., Franklin, TN (US)

(72) Inventors: DeepakKumar Yedlarajaiah, Naperville, IL (US); Daniel Wiens, Sparks, NV (US)

(21) Appl. No.: 18/761,156

(22) Filed: Jul. 1, 2024

Related U.S. Application Data
(63) Continuation of application No. 16/833,059, filed on Mar. 27, 2020.

(51) Int. Cl. G06Q 30/0283; G06Q 30/0234; G06Q 30/0601; G06Q 40/08; G16H 40/00

(52) U.S. Cl. CPC G06Q 30/0283; G06Q 30/0611; G06Q 40/08; G16H 40/00; G06Q 30/0234

(57) ABSTRACT

Quantitative estimate accuracy scoring is provided. An estimate scoring engine uses estimate scoring model logic to quantitatively measure the accuracy of an estimate for healthcare services relative to actual values associated with an adjudication of a claim for the services. Scores are determined for various factors that impact estimate accuracy, and a total estimate accuracy score may be determined. A report may be generated and displayed in a user interface including information received and/or determined by the estimate scoring engine. A plurality of estimates may be evaluated, and the report may include a summary and details associated with the accuracy of a range of estimates. An accuracy rate associated with the range of estimates may be measured against the accuracy rate of other healthcare services providers, payers, and/or estimation models in an ‘apples-to-apples’ comparison. Accordingly, issues associated with estimate inaccuracies may be identified and addressed to improve estimate accuracy.

### INCORPORATION BY REFERENCE TO ANY PRIORITY APPLICATIONS

This application is a continuation of U.S. patent application Ser. No. 16/833,059, filed Mar. 27, 2020, entitled “ESTIMATE ACCURACY SCORING MODEL,” which is incorporated herein by reference in its entirety for all purposes.

Any and all applications for which a foreign or domestic priority claim is identified in the Application Data Sheet as filed with the present application are hereby incorporated by reference under 37 CFR 1.57.

### BACKGROUND

When an individual seeks and/or receives healthcare services, the healthcare provider may determine and provide an estimate of the individual's out of pocket responsibility for the services. The estimate may be determined at or before the point of service by an estimation model utilized by the healthcare service provider (or by an agent associated with the provider). The estimation model may determine an estimated amount owed by the individual after insurance payments and real time insurance benefits are factored in. For a variety of reasons, the estimated amount may be inaccurate, wherein “accuracy” and its related adjective and adverbs refer to how closely the estimated amount is to the actual amount that the individual may owe after a claim has been adjudicated. Currently, there is not a quantitative way to score the many variables that can impact estimates and cause them to be inaccurate. Additionally, there is not a current way to analyze scored variables for determining one or more sources of inaccuracies that may be corrected for improving estimation model accuracy. Moreover, although an estimation accuracy goal may be set as an industry standard (e.g., 90% accuracy 85% of the time), currently, there is not a current method to provide an ‘apples-to-apples’ comparison between different revenue cycle vendors on estimate accuracy. Accordingly, the accuracy of various estimation models may not be accurately projected nor compared.

As can be appreciated, an absence of providing individuals with accurate estimate information prior to services being rendered can place a strain on the healthcare system, wherein the accuracy of an estimate may be of importance to both the individual and the healthcare services provider. For example, if an actual owed amount is higher than the estimate, the individual may be disgruntled/frustrated with the healthcare services provider. In some examples, the individual may be unable or unwilling to pay the owed amount, wherein rendered healthcare services do not provide a security or other object that can be repossessed in the event of a default. Accordingly, the healthcare provider may increasingly hold onto larger accounts receivable for longer periods of time as individuals delay in making payments for procedures, or even default on payments for procedures. As another example, if an actual owed amount is less than the estimate, the healthcare provider may need to issue a refund, which may be associated with additional expenses for the healthcare provider. Healthcare providers (or their agents) currently use various computer systems to perform various processes to provide healthcare services and to bill for those services. As will be appreciated, if the accuracy of the individual systems that perform these processes were to be improved (e.g., such as an estimation model that generates estimates), the overall system itself and the quality of care available to patients can be improved.

### SUMMARY

The present disclosure provides a system, method, and a computer readable storage device for providing quantitative estimate accuracy scoring. Aspects of the present disclosure can be utilized to generate an estimate accuracy report that indicates how accurate an estimate is based on a comparison against an associated claim and payment information. The system includes an estimate scoring engine configured to implement estimate scoring logic to quantitatively score various elements associated with different variables/factors that impact the estimate accuracy. By providing a standardized and quantitative method of measuring estimate accuracy, healthcare services providers are enabled to compare, in an ‘apples-to-apples’ comparison, different revenue cycle vendors on their claims of estimate accuracy. Aspects of the present disclosure provide a standardized, accurate, intuitive, and easy way to compare the estimate accuracy across vendors and payers. In some examples, scores generated by the system enable an identification of different reasons why an estimate may be less accurate. Accordingly, issues associated with estimate inaccuracies may be identified and addressed to improve estimate accuracy. As can be appreciated improved estimate accuracy helps to accurately inform patients of their financial responsibility and can enable the patients to ensure financial readiness prior to the service. Accordingly, patients are less likely to default on payment of their responsibility amount, which can increase the efficiency of billing processes.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The accompanying drawings, which are incorporated in and constitute a part of this disclosure, illustrate various aspects and examples of the present invention. In the drawings:

FIG. 1 is a block diagram illustrating an example operating environment for implementing aspects of the present disclosure;

FIG. 2 is a block diagram illustrating components of an estimate scoring model system with which aspects of the present disclosure may be practiced;

FIGS. 3A-C illustrate an example user interface as may be displayed to a user for providing estimate scoring information and functionalities;

FIG. 4 is a flow chart showing general stages involved in an example method for providing quantitative estimate scoring; and

FIG. 5 is a block diagram illustrating physical components of an example computing device with which aspects may be practiced.

### DETAILED DESCRIPTION

The following detailed description refers to the accompanying drawings. Wherever possible, the same reference numbers are used in the drawings and the following description to refer to the same or similar elements. While aspects of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the present disclosure, but instead, the proper scope of the present disclosure is defined by the appended claims. Examples may take the form of a hardware implementation, or an entirely software implementation, or an implementation combining software and hardware aspects. The following detailed description is, therefore, not to be taken in a limiting sense.

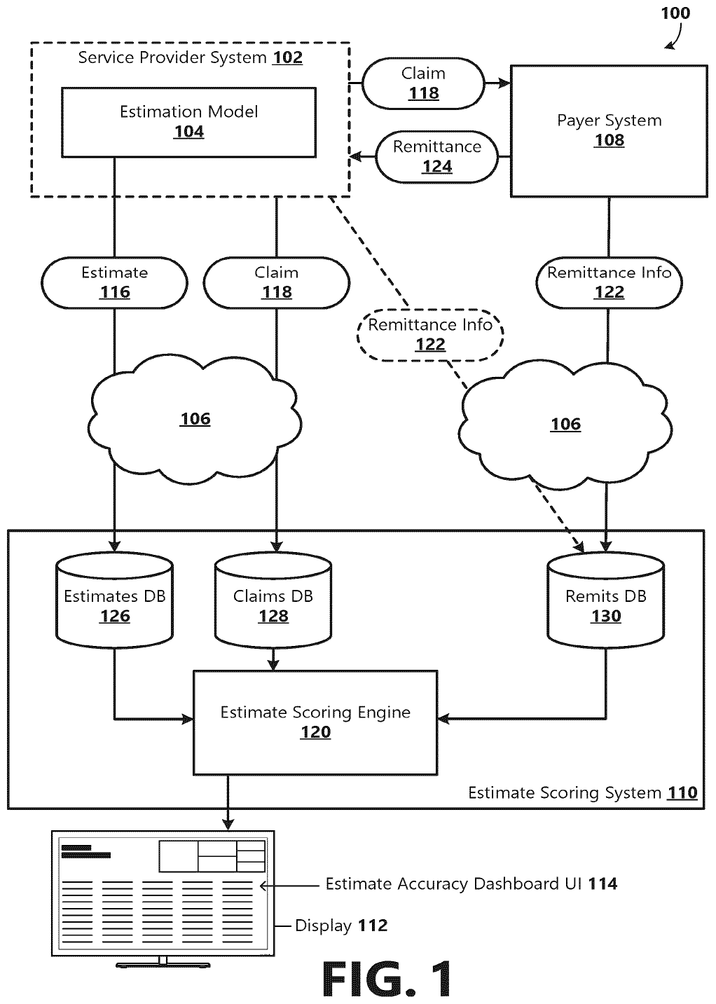

The present disclosure provides a system, method, and computer readable storage device including computer readable instructions, which when executed by a processing unit, provide quantitative estimate accuracy scoring for improving estimate accuracy. Although examples are given herein primarily involving healthcare providers and patients, it will be recognized that the present disclosure is applicable to other types of services that provide estimates to clients. As such, the terms “patient” and “client” may be used interchangeable herein. FIG. 1 is a block diagram illustrating an example operating environment 100 in which an estimate scoring system 110 may be implemented and quantitative estimate accuracy scoring functionalities can be performed. In example aspects, the estimate scoring system 110 includes an estimate scoring engine 120 operative or configured to utilize a quantitative method to measure the accuracy of an estimate 116 and provide standardized measurements that can be used to easily and intuitively compare estimate accuracy across different revenue cycle vendors and payers. As used herein, “accuracy” and its related adjectives and adverbs do not refer to the correctness of how calculations are preformed (which are assumed to be performed correctly, unless stated otherwise), but refer to how close an estimate 116 is to a final value. For example, if the final value for the costs of healthcare services is $100, an estimate of $99 would be more accurate than an estimate 116 of $98. Quantitative estimate accuracy measurements determined by the estimate scoring engine 120 may provide healthcare providers an ‘apples-to-apples’ comparison between different revenue cycle vendors on their claims of estimate accuracy. Additionally, quantitative estimate accuracy measurements may be used to make various determinations. For example, quantitative estimate accuracy measurements may be used to identify different reasons why an estimate 116 may not be accurate, which can enable the healthcare provider or their agent to tune their estimation model or implement other measures to improve accuracy.

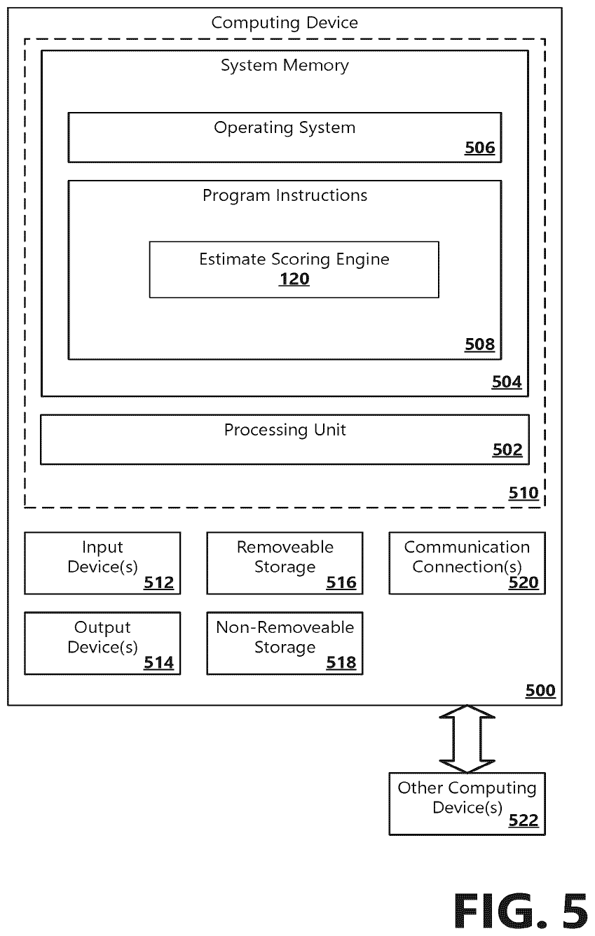

With reference to FIG. 1, the example operating environment 100 includes a service provider system 102, a payer system 108, and the estimate scoring system 110. Each of the systems 102, 108, 110 includes one or more computing devices and one or more data storage devices, and is in communication with a network 106 or a combination of networks for exchanging data to determine and provide quantitative estimate accuracy information. The one or more computing devices are illustrative of a wide variety of computing devices, the hardware of which is discussed in greater detail in regard to FIG. 5. Non-limiting examples of computing devices include server computers, desktop computers, laptop computers, mobile computing devices, wearable computing devices, and the like. The network 106 or combination of networks may include any type of public or private data network for communicating data between computer systems within a same enterprise and/or amongst various entities at different geographic locations. The Internet is one example of one possible network 106.

According to an aspect, the service provider system 102 is illustrative of an information system utilized by a healthcare services provider (or an agent of the healthcare services provider, such as a clearinghouse) configured to store and process clinical, financial, and administrative data as part of providing healthcare services to patients. The example operating environment 100 may include a plurality of service provider systems 102 associated with a plurality of healthcare services providers. The service provider system 102 may include or be operatively connected to an estimation model 104 operative or configured to create estimates 116 of services for patients before or at the point-of-service. An estimate 116 may include an estimated amount owed by a patient for healthcare services after insurance payments and real time insurance benefits are factored in. For example, the estimation model 104 may create an estimate 116 by utilizing data from the healthcare service provider's charge description master (CDM), claims history, payer contract terms, and the patient's insurance benefits. The CDM may include a listing of individual procedures, services and items that are billable to a patient or insurance provider. Payer contract terms may be based on an agreement between the healthcare services provider and the payer. The estimation model 104 may further incorporate financial assistance policies for self-pay patients. According to an aspect, the estimate scoring system 110 is configured to receive estimates 116 generated by the service provider system 102. In some examples, when an estimate 116 is generated by the service provider system 102, the estimate 116 is transmitted to and stored in an estimates database 126 where it can be accessed by the estimate scoring engine 120.

When services are rendered to the patient, a claim 118 may be generated by the service provider system 102 that includes healthcare claim billing information associated with the services rendered to the patient. For example, the claim 118 may include a patient description (e.g., patient demographic information), information about a condition for which the patient was treated, the services that were provided, and costs of the treatment. The service provider system 102 may submit the claim 118 to a designated payer system 108. In some examples, the claim 118 may be submitted to the payer system 108 as an electronic data file (e.g., electronic data interchange (EDI) 837 transaction set). For example, the EDI 837 transaction set is a format established to meet Health Insurance Portability and Accountability Act (HIPAA) requirements for the electronic submission of healthcare claim information. According to an aspect, the estimate scoring system 110 is configured to receive claims 118 generated by the service provider system 102. In some examples, when a claim 118 is generated by the service provider system 102, the claim 118 is transmitted to and stored in a claims database 128 where it can be accessed by the estimate scoring engine 120.

The payer system 108 may be configured to receive and process the claim 118. When the claim 118 is approved, remittance 124 of the payer's portion of the costs of the treatment may be made to the service provider system 102 based on a pre-negotiated amount or percentage (e.g., according to the payer contract terms). In some examples, the remittance 124 may be submitted to the service provider system 102 electronically (e.g., in an EDI 835 transaction set). For example, the EDI 835 transaction set may be used to auto-post a claim payment and/or send an explanation of payments (EOP) to the service provider. According to an aspect, the estimate scoring system 110 is configured to receive remittance information 122 from the payer system 108 or from the service provider system 102, wherein the remittance information 122 includes an indication of the patient's true liability for the rendered services. The remittance information 122 may further include details of the claim payment, such as: what charges were paid, reduced, or denied; deductible, coinsurance, and/or copayment information; any bundling or splitting of claims or line items; and how the claim payment was made. In some examples, the remittance information 122 is provided to the estimate scoring system 110 in the EDI 835 transaction set. The remittance information 122 may be stored in a remittance database 130 where it can be accessed by the estimate scoring engine 120.

The estimates database 126, claims database 128, and remittance database 130 are illustrative of one or more data storage devices or computer readable storage media on which various information are stored. In some examples, the estimates database 126, claims database 128, and remittance database 130 can be cloud-based storage systems that are separate and remote from the estimate scoring engine 120, but are in communication with the estimate scoring engine 120 through the network 106.

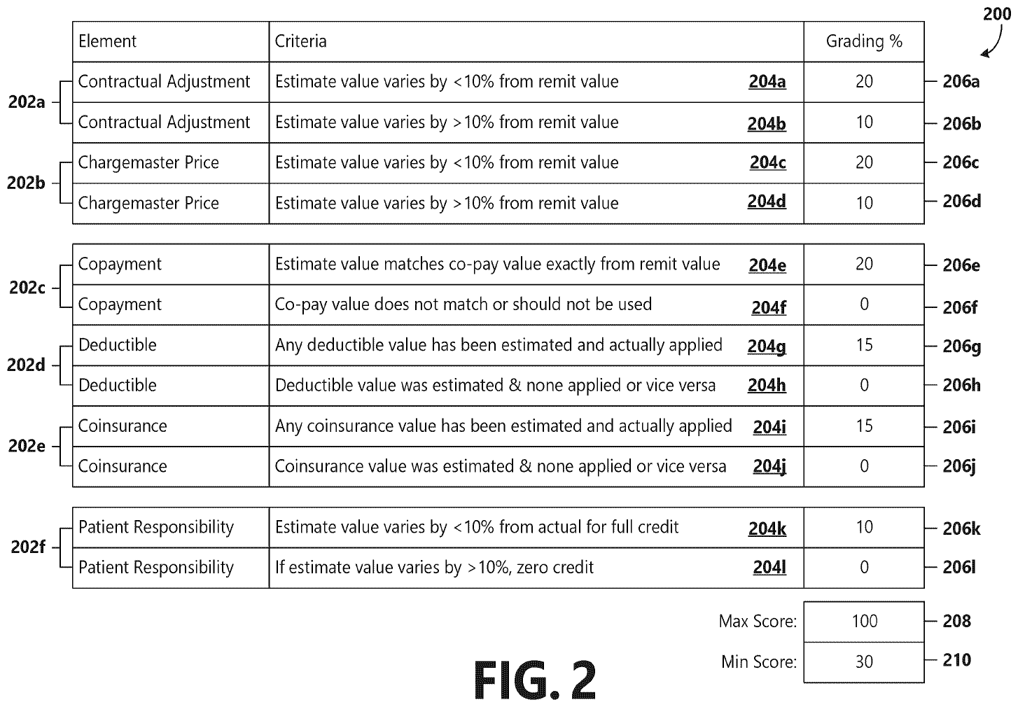

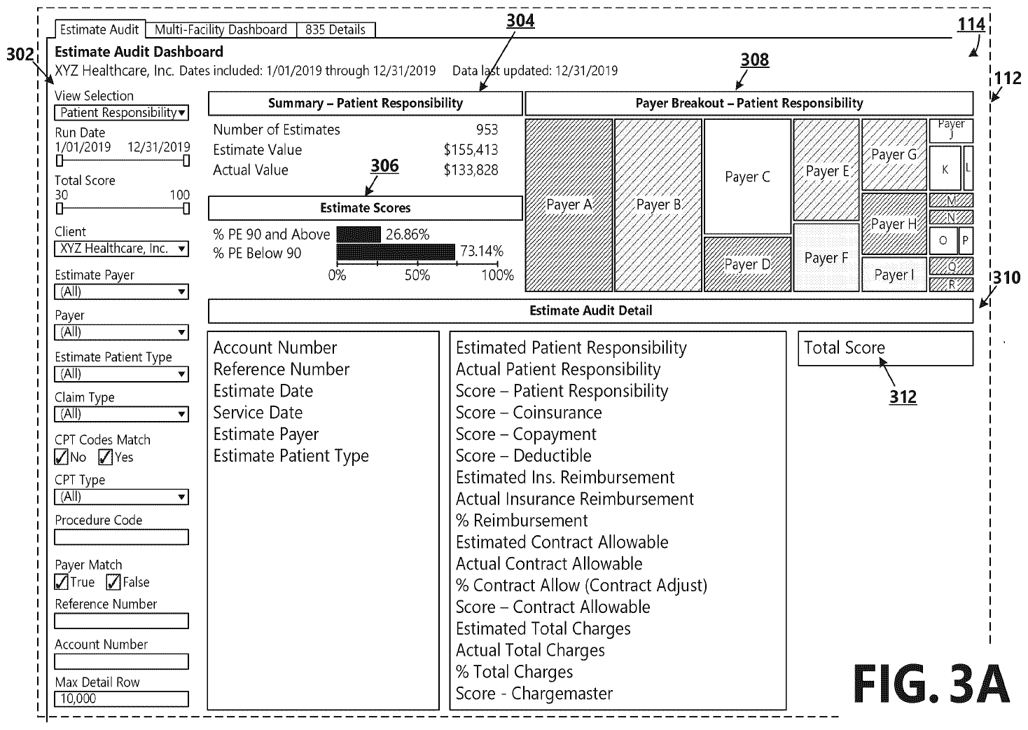

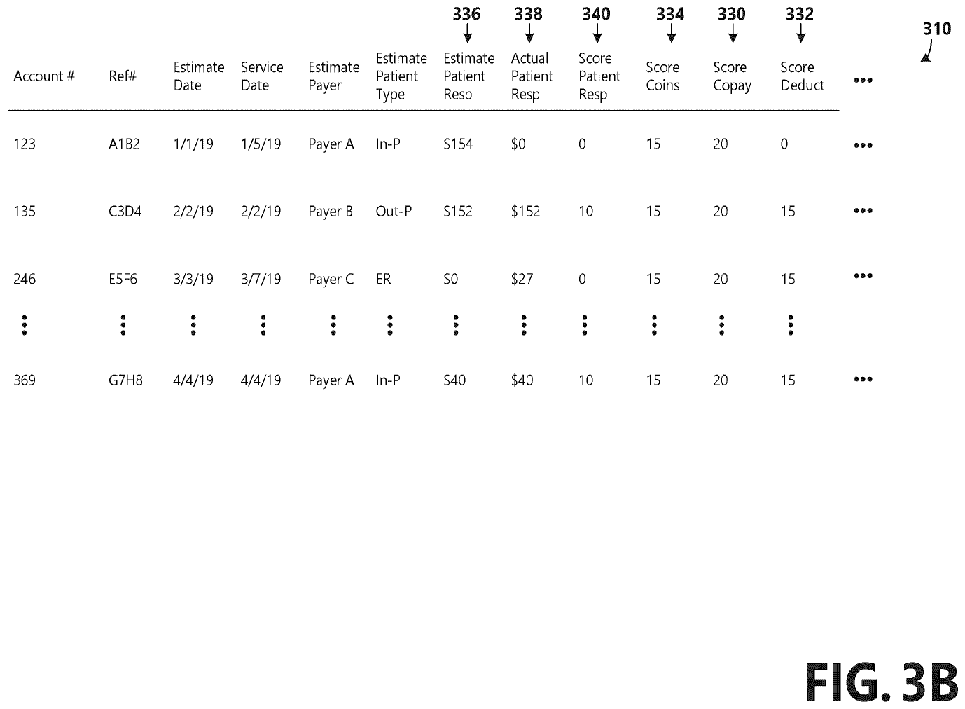

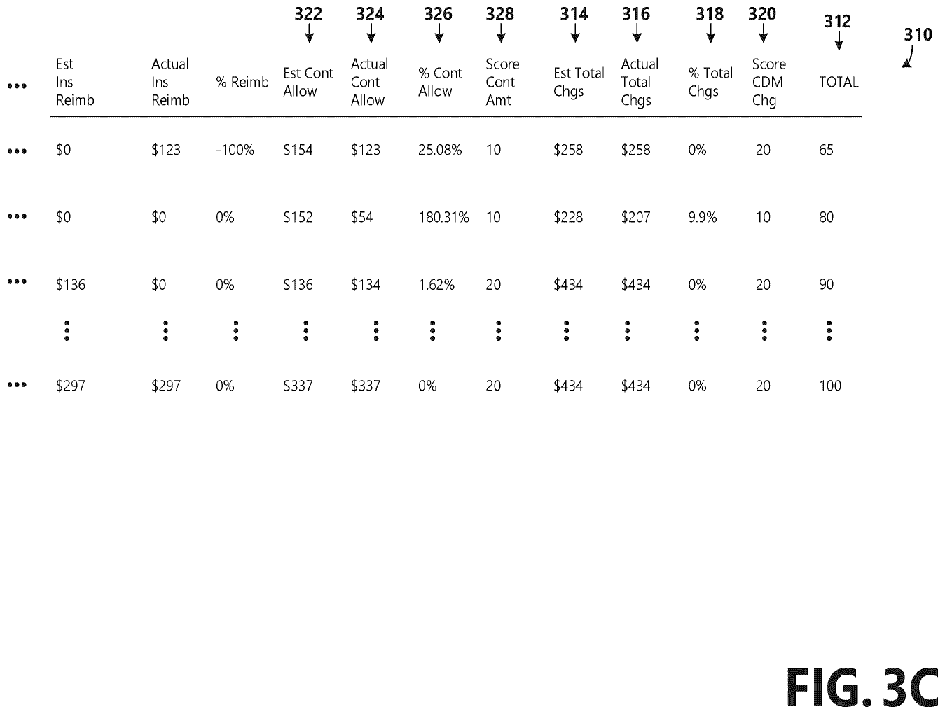

According to an aspect, the estimate scoring engine 120 is illustrative of a software application, module, or computing device configured to match an estimate 116 to a claim 118 and to a remittance 124, apply estimate scoring model logic to compare and score various factors/elements associated with the estimate 116, and to determine an overall accuracy score for the estimate 116 based on the element scores. Instructions for the estimate scoring engine 120 can be executed by a single computing device or can be distributed across a plurality of computing devices that are in communication with each other and form a part of the estimate scoring system 110. The estimate scoring model logic, which is described below with reference to FIG. 2, gives weightage for elements that comprise the accuracy calculation, thus providing a quantitative and standardized way to measure the accuracy of the estimate 116. Accordingly, the accuracy of the estimate 116 can be quantified and compared across estimation models 104, service providers, and payers. According to an aspect, the estimate scoring engine 120 is further operative or configured to generate an estimate accuracy dashboard UI 114 that can be displayed on a display device 112 and used to display information received and/or determined by the estimate scoring engine 120 and for enabling a user to interact with functionalities provided by the dashboard UI 114 through a manipulation of graphical icons, visual indicators, and the like. Aspects of the dashboard UI 114 will be described in further detail below with reference to FIGS. 3A-C.

With reference now to FIG. 2, an example embodiment of estimate scoring model logic 200 utilized by the estimate scoring engine 120 for measuring the accuracy of the estimate 116 is illustrated. According to an aspect, the estimate scoring model logic 200 defines a plurality of elements 202a-f (generally 202) and scoring criteria 204a-l (generally 204) and grading percentages 206a-l (generally 206) for each element 202. As should be appreciated, in some examples, the scoring criteria 204 and/or grading percentages 206 may vary from the specific scoring criteria and grading percentages included in the example estimate scoring model logic 200 illustrated in FIG. 2 and described herein. The elements 202 are associated with factors or variables that have been determined to impact estimate accuracy. In the example embodiment, the elements 202 include a contractual adjustment element 202a, a chargemaster (i.e., CDM) price element 202b, a copayment element 202c, a deductible element 202d, a coinsurance element 202e, and a patient responsibility element 202f. In other examples, additional and/or alternative elements 202 may be included. The estimate scoring engine 120 may be configured to evaluate each element 202 based on the estimate 116, claim 118, and remittance information 122, apply the scoring criteria 204, calculate a score for each element based on the grading percentage 206, and total the element scores to determine a total accuracy score for the estimate 116. In some examples, a maximum possible score 208 is 100, and a minimum possible score 210 is 30. According to an aspect, based on a determined criticality of an element 202 to the accuracy of an estimate 116, some elements 202 may receive credit for exact matches only, while other elements 202 may receive partial credit for variances.

As an illustrative example, consider that a patient has insurance coverage with insurance provider/payer ABC and goes to a healthcare services provider for an X-ray of his left hand. The patient may, before or at the point of service, receive an estimate 116 from the healthcare services provider for the patient's financial responsibility amount for the X-ray. The healthcare services provider (or the service provider's agent) may use an estimation model 104 to generate the estimate 116 based on the healthcare services provider's CDM, contract terms with payer ABC, and the patient's insurance benefits. For example, if the CDM price for the X-ray is $100 (e.g., the price that a patient without insurance coverage may be charged) and the contract terms include a discount of 40%, the contractual adjustment amount is $60. The estimation model 104 may determine the patient's responsibility amount based on a copayment, deductible, and coinsurance associated with the patient's benefits and depending on an amount the patient has paid for healthcare services towards a deductible amount prior to the estimate 116 being generated. In some examples, this copayment, deductible, and coinsurance information may be provided to the service provider system 102 by the payer system 108 in a Healthcare Eligibility, Coverage and Benefit Response (EDI 271 response) that includes details of coverage, benefits, and eligibility in response to a Healthcare Eligibility, Coverage and Benefit Request (EDI 270 request) sent by the service provider system 102 to the payer system 108. For example, the patient may be responsible for the full $60 or for a portion of the $60 based on whether the patient has met his deductible and whether the patient has a copayment or coinsurance. In this illustrative example, the patient may have met his deductible and has a copayment of $30. Accordingly, the estimated payer's responsibility may be determined to be $30 and the estimated patient's responsibility may be determined to be $30. The estimate 116 may be provided to the estimate scoring system 110 and stored in the estimates database 126. After the X-ray is performed, a claim 118 for the procedure may be sent to the payer system 108 where it may be processed and a remittance 124 may be sent to the healthcare services provider. The claim 118 and remittance information 122 may be provided to the estimate scoring system 110 and stored in the claims database 128 and the remittance database 130 respectively.

According to the example estimate scoring model logic 200 illustrated in FIG. 2, the estimate scoring engine 120 may determine a score for the contractual adjustment price element 202a by matching the contractual adjustment value determined for the estimate 116 against the actual contractual adjustment value included in the remittance information 122. According to a first scoring criterion 204a associated with the contractual adjustment price element 202a, if the estimated contractual adjustment value varies by less than 10% from the actual contractual adjustment value, a full score may be determined for the contractual adjustment price element 202a. For example, based on a grading percentage 206a of 20%, if the estimated contractual adjustment value varies by less than 10% from the actual contractual adjustment value, the element score for the contractual adjustment price element 202a may be determined to be 20. According to a second scoring criterion 204b associated with the contractual adjustment price element 202a, if the estimated contractual adjustment value varies by greater than 10% from the actual contractual adjustment value, a partial score may be determined for the contractual adjustment price element 202a, which allows for a variance in the accuracy of the contractual adjustment price. For example, based on a grading percentage 206b of 10%, if the estimated contractual adjustment value varies by greater than 10% from the actual contractual adjustment value, the element score for the contractual adjustment price element 202a may be determined to be 10.

According to an aspect, using the example estimate scoring model logic 200, the estimate scoring engine 120 may further determine a score for the chargemaster (i.e., CDM) price element 202b by matching the CDM value included in the estimate 116 against the actual CDM value specified in the remittance information 122. According to a first scoring criterion 204c associated with the CDM price element 202b, if the estimated CDM value varies by less than 10% from the actual CDM value, a full score may be determined for the CDM price element 202b. For example, based on a grading percentage 206c of 20%, if the estimated CDM value varies by less than 10% from the actual CDM value, the element score for the CDM price element 202b may be determined to be 20. According to a second scoring criterion 204d associated with the CDM price element 202b, if the estimated CDM value varies by greater than 10% from the actual CDM value, a partial score may be determined for the CDM price element 202b, which allows for a variance in the accuracy of the CDM price. For example, based on a grading percentage 206d of 10%, if the estimated CDM value varies by greater than 10% from the actual CDM value, the element score for the CDM price element 202b may be determined to be 10.

According to an aspect, using the example estimate scoring model logic 200, the estimate scoring engine 120 may further determine a score for various benefit elements: the copayment element 202c, the deductible element 202d, and the coinsurance element 202e. For example, the estimate scoring engine 120 may determine a score for the copayment element 202c by matching the copayment value included in the estimate 116 against the actual copayment value specified in the remittance information 122. According to a first scoring criterion 204e associated with the copayment element 202c, if the estimated copayment value matches the actual copayment value, a full score may be determined for the copayment element 202c. For example, based on a grading percentage 206e of 20%, if the estimated copayment value matches the actual copayment value, the element score for the copayment element 202c may be determined to be 20. According to a second scoring criterion 204f associated with the copayment element 202c, if the estimated copayment value does not match the actual copayment value, if a copayment value is included in the estimate 116 but a copayment should not be used, or if a copayment value is missing, no credit may be given, and based on a grading percentage 206f of 0%, a score of 0 may be determined for the copayment element 202c.

According to an aspect, using the example estimate scoring model logic 200, the estimate scoring engine 120 may determine a score for the deductible element 202d by determining if any deductible value is included in the estimate 116 and whether any deductible value is actually applied according to the remittance information 122. According to a first scoring criterion 204g associated with the deductible element 202d, if any deductible value is estimated and included in the estimate 116 and if any deductible value is actually applied, a full score may be determined for the deductible element 202d. For example, based on a grading percentage 206g of 15%, if a deductible value is estimated and actually applied, the element score for the deductible element 202d may be determined to be 15. According to an aspect, by assigning a full score for the deductible element 202d if any deductible is actually applied, the service provider may not be penalized for an amount that the patient may have paid towards their deductible between the time the estimate 116 was created and the time the services were rendered. According to a second scoring criterion 204h associated with the deductible element 202d, if a deductible value is estimated but no deductible is actually applied or if a deductible value is not estimated but a deductible is actually applied, no credit may be given, and based on a grading percentage 206h of 0%, a score of 0 may be determined for the deductible element 202d.

According to an aspect, using the example estimate scoring model logic 200, the estimate scoring engine 120 may determine a score for the coinsurance element 202e by matching the coinsurance value included in the estimate 116 against the actual coinsurance value specified in the remittance information 122. According to a first scoring criterion 204i associated with the coinsurance element 202e, if any coinsurance value is estimated and is actually applied, a full score may be determined for the coinsurance element 202e. For example, based on a grading percentage 206i of 20%, if any coinsurance value is estimated and is actually applied, the element score for the coinsurance element 202e may be determined to be 15. According to a second scoring criterion 204j associated with the coinsurance element 202e, if a coinsurance value is estimated but no coinsurance is actually applied or if a coinsurance value is not estimated but a coinsurance is actually applied, no credit may be given, and based on a grading percentage 206j of 0%, a score of 0 may be determined for the coinsurance element 202e.

According to an aspect, using the example estimate scoring model logic 200, the estimate scoring engine 120 may further determine a score for the patient responsibility element 202f by matching the patient responsibility value included in the estimate 116 against the actual patient responsibility value specified in the remittance information 122. According to a first scoring criterion 204k associated with the patient responsibility element 202f, if the estimated patient responsibility value varies by less than 10% from the actual patient responsibility value, a full score may be determined for the patient responsibility element 202f. For example, based on a grading percentage 206k of 10%, if the estimated patient responsibility value varies by less than 10% from the actual patient responsibility value, the element score for the patient responsibility element 202f may be determined to be 10. According to a second scoring criterion 2041 associated with the patient responsibility element 202f, if the estimated patient responsibility value varies by greater than 10% from the actual patient responsibility value, no credit may be given, and based on a grading percentage 2061 of 0%, a score of 0 may be determined for the patient responsibility element 202f.

According to an aspect, using the example estimate scoring model logic 200, the estimate scoring engine 120 may total the element scores to determine a total accuracy score for the estimate 116. Accordingly, the variables that impact the accuracy of estimates 116 may be quantified and estimate accuracy across different revenue cycle vendors may be compared according to a standardized scoring method. According to an aspect, the dashboard UI 114 generated by the estimate scoring engine 120 includes a display of information received and/or determined by the estimate scoring engine 120. For example, for an estimate 116, the dashboard UI 114 may include the element 202 values compared by the estimate scoring engine 120 using the estimate scoring model logic 200, the determined element scores, and the determined total accuracy score. With reference now to FIGS. 3A-C, an example embodiment of the dashboard UI 114 is illustrated. For example, the dashboard UI 114 may be displayed on a display device 112 of a computing device for displaying an estimate accuracy report including information received and/or determined by the estimate scoring engine 120 and for enabling a user to interact with functionalities provided by the dashboard UI 114 through a manipulation of graphical icons, visual indicators, and the like. As illustrated in the example, the dashboard UI 114 may include various filtering options 302 that enable the user to view different data. Example filtering options 302 may include: a filtering option to view summary and details associated with the accuracy of various estimated values versus actual values, such as patient responsibility values, coinsurance values, copayment values, deductible values, insurance/payer reimbursement values, contract allowable values, etc.; a filtering option to select a date range of estimates 116 and a total accuracy score 312 range to view or analyze summaries and details associated with the accuracy of various estimated values versus actual values within the selected range(s); filtering options to select to view/analyze summaries and details associated with the accuracy of various estimated values versus actual values associated with: one or more clients/healthcare services providers, one or more estimated payers, one or more actual payers, one or more patient types (e.g., inpatient or outpatient), one or more claim types, matching and/or non-matching Current Procedural Terminology (CPT) codes (e.g., matched between an estimate 116, a claim 118, and/or a remittance 124), one or more procedure codes, matching and/or non-matching payers (e.g., matched between an estimate 116, a claim 118, and/or a remittance 124), a particular reference number (e.g., internal estimate 116 identifier), and/or a particular account number (e.g., healthcare services provider-specific or payer-specific identifier for an estimate 116); a filtering option to select a maximum amount of detail to display for a matched estimate; etc. As should be appreciated, fewer, additional, and/or alternative filtering options 302 may be included and are within the scope of the present disclosure.

In some examples, the dashboard UI 114 may include a summary section 304 that includes a summary of a particular value. For example, the particular value may be associated with a selected value type (e.g., selected via a selection of a filtering option 302 associated with the value type). In the example dashboard UI 114 illustrated in FIG. 3A, the summary section 304 includes a summary of patient responsibility values. The summary may include a number of estimates 116 matched (i.e., with a claim 118 and a remittance 124) and analyzed, a total estimate value (e.g., patient responsibility estimate value) associated with the matched and analyzed estimates 116, and a total actual value (e.g., patient responsibility actual value) associated with the matched and analyzed estimates 116. For example, in the example dashboard UI 114, the summary includes 953 matched estimates 116, wherein the estimated patient responsibility value for the 953 estimates is $155,413 and the actual patient responsibility value for the 953 estimates is $133,828 for a difference of $21,585 between the estimated and actual values. As should be appreciated, fewer, additional and/or alternative summary information may be included in the summary section 304 and are within the scope of the present disclosure.

In some examples, the dashboard UI 114 may further include an estimate scores section 306 that includes a display of a percentage of the matched estimates 116 that have a total accuracy score 312 that meet or exceed a determined acceptable accuracy rate and a display of a percentage of the matched estimates 116 that have a total accuracy score 312 that fall below the determined acceptable accuracy rate. For example, an acceptable accuracy rate may be determined as an industry standard. One example acceptable accuracy rate is 90% accuracy 85% of the time. As mentioned previously, currently, without an implementation of aspects of the present disclosure, there is not a standardized way to compare accuracy rates across different healthcare services providers or revenue cycle vendors. The estimate scores section 306 of the dashboard UI 114 may provide a quick and intuitive view of an entity's performance in relation to meeting the acceptable accuracy rate. In the illustrated example, the selected healthcare services provider has an accuracy rate of 90% accuracy 26.86% of the time. Using aspects of the present disclosure, the healthcare services provider's accuracy rate (or an estimation model's accuracy rate) may be compared in an ‘apples-to-apples’ comparison against another healthcare services provider's or another estimation model's accuracy rate.

In some examples, the dashboard UI 114 may further include a payer breakout section 308, which may include a visual indication of a difference between estimated and actual values for various payers. For example, each payer associated with the matched/analyzed estimates 116 may be represented as a box. According to an aspect, the size of each payer box may represent the number of estimates 116 associated with that payer, wherein a larger box may indicate a larger number of estimates 116 and a smaller box may indicate a fewer number of estimates 116. According to another aspect, color coding may be utilized to indicate how far above or below the estimated values are to the actual values for the payer. As one example, a box may be displayed in red to indicate that the estimated values for the associated payer are higher than the actual values, and a box may be displayed in blue to indicate that the estimated values for the associated payer are lower than the actual values. As should be appreciated, the usage of red and blue as indicators is exemplary; other colors or visual indicators (e.g., patterns, hatching, shading) may be used and are within the scope of the present disclosure. Additionally, in some examples, shading or color intensity may be utilized to indicate how far above or below the estimated values are from the actual values. For example, a darker shading or more intense color may be used to indicate that estimated values for the associated payer are farther away from the actual values, wherein a lighter shading or less intense color may be used to indicate that the estimated values for the associated payer are closer to the actual values. In some examples, the boxes may be selectable. For example, when a box is selected, the dashboard UI 114 may be updated to show details of the data received and/or determined by the estimate scoring engine 120 in association with that payer.

In some examples, the dashboard UI 114 may further include an estimate audit details section 310, which may include details about each matched estimate 116. For example, the details may include data received and/or determined by the estimate scoring engine 120 in association with a matched estimate 116. Examples of data associated with a matched estimate 116 that may be included in the estimate audit details section 310 include a healthcare services provider-specific account number, an internal (i.e., an estimate scoring engine-specific) reference number, an estimate date (i.e., the date the estimate 116 was created), a service date (i.e., the date the healthcare services were rendered), a payer used for creating the estimate 116, a patient type used for creating the estimate 116, the estimated patient responsibility value, the actual patient responsibility value, the determined patient responsibility element 202f score, the determined coinsurance element 202e score, the determined copayment element 202c score, the determined deductible element 202d score, the estimated insurance/payer reimbursement value, the actual insurance/payer reimbursement value, the percent variation between the estimated and actual insurance/payer reimbursement value, the estimated contractual adjustment value, the actual contractual adjustment value, the percent variation between the estimated and actual contractual adjustment value, the determined contractual adjustment element 202a score, the estimated total charges, the actual total charges, the percent variation between the estimated and actual total charges, the determined chargemaster price element 202b score, and the total accuracy score 312 for the estimate 116. An exploded view of example data that may be included in the estimate audit details section 310 is illustrated in FIGS. 3B and 3C. As should be appreciated, fewer, additional, and/or alternative data may be included in the estimate audit details section 310.

As mentioned previously, using aspects of the present disclosure, the healthcare services provider's accuracy rate (or an estimation model's accuracy rate) may be compared in an ‘apples-to-apples’ comparison against another healthcare services provider's or another estimation model's accuracy rate. Additionally, an accuracy rate below a target or acceptable accuracy rate may be an indication of an issue with an estimation process used by the service provider system 102 or with the data used by the estimation model 104. An analysis of the details (e.g., included in the estimate audit details section 310) may be performed to determine the root cause or source of inaccuracies. For example, the data included in the estimate audit details section 310 may be analyzed for identifying patterns of inaccuracies that indicate a root cause or source of the inaccuracies. As an example, if copayment element 202c scores are repeatedly 0, a determination may be made that the way copayment information is determined for an estimate 116 is errant and needs to be examined and tweaked. As another example, if estimated total charges are recurrently inaccurate, a determination may be made that there is a problem with the way the service provider system 102 (or a user of the service provider system) selects procedures, services, or billable items for inclusion in an estimate 116 or the chargemaster used by the estimation model 104 is out-of-date or otherwise inaccurate. Accordingly, the healthcare services provider may be enabled to address the identified root cause or source of inaccuracies and improve the accuracy of estimates 116.

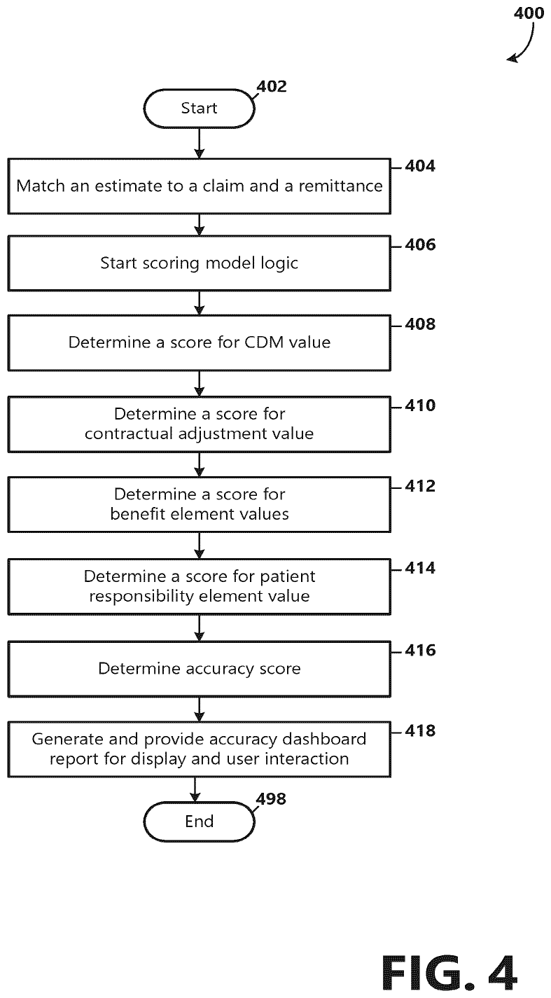

FIG. 4 illustrates a flow chart showing general stages involved in an example method 400 for providing quantitative estimate scoring. The method 400 begins at START OPERATION 402 and proceeds to OPERATION 404, where an estimate 116 for services may be matched to a claim 118 for the rendering of the services and a remittance 124 made by a payer system 108 in satisfaction of the claim 118. For example, the estimate 116, claim 118, and remittance information 122 associated with the remittance 124 may be provided to the estimate scoring system 110 in real-time as they are generated or in batches. When an estimate 116 is matched to a claim 118 and to a remittance 124, at OPERATION 406, the estimate scoring engine 120 may use the estimate scoring logic 200 to quantitatively measure the accuracy of the estimate 116.

At OPERATION 408, using the estimate scoring logic 200, a CDM price element 202b score may be determined by matching the CDM value included in the estimate 116 against the actual CDM value specified in the remittance information 122 and applying the scoring criteria 204. For example, if the estimated CDM value varies by less than 10% from the actual CDM value, a full score (e.g., 20) may be determined for the CDM price element 202b, and if the estimated CDM value varies by greater than 10% from the actual CDM value, a partial score (e.g., 10) may be determined for the CDM price element 202b, which allows for a variance in the accuracy of the CDM price.

For example and with reference to the example values included in the example estimate audit details section 310 of the dashboard UI 114 illustrated in FIGS. 3B and 3C for account number 123, the CDM value included in the estimate 116 may be $258. This value may be referred to as the estimated total charges 314 in the estimate audit details section 310. The CDM value included in the remittance information 122 may be $258. This value may be referred to as the actual total charges 316 in the estimate audit details section 310. Accordingly, the percentage difference between the estimated and actual values is 0% (referred to in the estimate audit details section 310 as % total charges 318). Based on the estimate scoring logic 200, since the estimated CDM value varies by less than 10% from the actual CDM value, the CDM price element score 320 may be a full score of 20.

At OPERATION 410, using the estimate scoring logic 200, a contractual adjustment element 202a score may be determined by matching the contractual adjustment value determined for the estimate 116 against the actual contractual adjustment value included in the remittance information 122. For example, if the estimated contractual adjustment value varies by less than 10% from the actual contractual adjustment value, a full score (e.g., 20) may be determined for the contractual adjustment price element 202a, and if the estimated contractual adjustment value varies by greater than 10% from the actual contractual adjustment value, a partial score (e.g., 10) may be determined for the contractual adjustment price element 202a, which allows for a variance in the accuracy of the contractual adjustment price.

For example and with reference again to the example values included in the example estimate audit details section 310 of the dashboard UI 114 illustrated in FIGS. 3B and 3C for account number 123, the contractual adjustment value included in the estimate 116 may be $154. This value may be referred to as the estimated contractual allowance 322 in the estimate audit details section 310. The contractual adjustment value included in the remittance information 122 may be $123. This value may be referred to as the actual contractual allowance 324 in the estimate audit details section 310. Accordingly, the percentage difference between the estimated and actual values is 25.08% (referred to in the estimate audit details section 310 as % contractual allowance 326). Based on the estimate scoring logic 200, since the estimated contractual adjustment value varies by more than 10% from the actual contractual adjustment value, the contractual adjustment price element score 328 may be a partial score of 10.

At OPERATION 412, using the estimate scoring logic 200, various benefit element values may be determined. In some examples, a score for the copayment element 202c may be determined by matching the copayment value included in the estimate 116 against the actual copayment value specified in the remittance information 122. For example, if the estimated copayment value matches the actual copayment value, a full score (e.g., 20) may be determined for the copayment element 202c, and if the estimated copayment value does not match the actual copayment value, if a copayment value is included in the estimate 116 but a copayment should not be used, or if a copayment value is missing, no credit may be given and a score of 0 may be determined for the copayment element 202c.

For example and with reference again to the example values included in the example estimate audit details section 310 of the dashboard UI 14 illustrated in FIGS. 3B and 3C for account number 123, an estimated copayment amount was estimated and actually applied. Accordingly, based on the estimate scoring logic 200, the copayment element score 330 is determined to be a full score of 20.

In some examples, a score for the deductible element 202d may be determined by determining if any deductible value is included in the estimate 116 and whether any deductible value is actually applied according to the remittance information 122. For example, if any deductible value is estimated and included in the estimate 116 and if any deductible value is actually applied, a full score (e.g., 15) may be determined for the deductible element 202d, and if a deductible value is estimated but no deductible is actually applied or if a deductible value is not estimated but a deductible is actually applied, no credit may be given and a score of O may be determined for the deductible element 202d.

For example and with reference again to the example values included in the example estimate audit details section 310 of the dashboard UI 114 illustrated in FIGS. 3B and 3C for account number 123, either a deductible value was estimated but no deductible was actually applied or a deductible value was not estimated but a deductible was actually applied. Accordingly, based on the estimate scoring logic 200, the deductible element score 332 is determined to be 0.

In some examples, a score for the coinsurance element 202e may be determined by matching the coinsurance value included in the estimate 116 against the actual coinsurance value specified in the remittance information 122. For example, if any coinsurance value is estimated and is actually applied, a full score (e.g., 15) may be determined for the coinsurance element 202e, and if a coinsurance value is estimated but no coinsurance is actually applied or if a coinsurance value is not estimated but a coinsurance is actually applied, no credit may be given and a score of O may be determined for the coinsurance element 202e.

For example and with reference again to the example values included in the example estimate audit details section 310 of the dashboard UI 114 illustrated in FIGS. 3B and 3C for account number 123, an estimated coinsurance amount was estimated and actually applied. Accordingly, based on the estimate scoring logic 200, the coinsurance element score 334 is determined to be a full score of 15.

At OPERATION 414, using the estimate scoring logic 200, a score for the patient responsibility element 202f may be determined by matching the patient responsibility value included in the For example, if the estimated patient responsibility value varies by less than 10% from the actual patient responsibility value, a full score (e.g., 10) may be determined for the patient responsibility element 202f, and if the estimated patient responsibility value varies by greater than 10% from the actual patient responsibility value, no credit may be given and a score of O may be determined for the patient responsibility element 202f.

For example and with reference again to the example values included in the example estimate audit details section 310 of the dashboard UI 114 illustrated in FIGS. 3B and 3C for account number 123, the patient responsibility value included in the estimate 116 may be $154. This value may be referred to as the estimated patient responsibility 336 in the estimate audit details section 310. The patient responsibility value included in the remittance information 122 may be $0. This value may be referred to as the actual patient responsibility 338 in the estimate audit details section 310. Accordingly, the percentage difference between the estimated and actual values is greater than 10% and based on the estimate scoring logic 200, the patient responsibility element score 340 may be 0.

At OPERATION 416, using the example estimate scoring model logic 200, a total accuracy score 312 may be determined for the estimate 116. For example, the total accuracy score 312 may be determined by totaling the element scores. Accordingly, the variables that impact the accuracy of estimates 116 may be quantified.

For example and with reference again to the example values included in the example estimate audit details section 310 of the dashboard UI 114 illustrated in FIGS. 3B and 3C for account number 123, the CDM price element score 320 (20), the contractual adjustment price element score 328 (10), the copayment element score 330 (20), the deductible element score 332 (0), the coinsurance element score 334 (15), and the patient responsibility element score 340 (0) may be totaled to determine the total accuracy score. In the illustrated example, the total accuracy score 312 for the estimate is 65.

At OPERATION 418, an estimate accuracy dashboard UI 114 may be generated and for displaying an estimate accuracy report including information received and/or determined by the estimate scoring engine 120 and for enabling a user to interact with functionalities provided by the dashboard UI 114 through a manipulation of graphical icons, visual indicators, and the like. In some examples, as part of generating the report of information for inclusion in the dashboard UI 114, the estimate scoring engine 120 may apply the estimate scoring model logic 200 to a plurality of matched estimates 116, claims 118, and remittance information 122 for generating a summary and details associated with the accuracy of a range of estimates 116. In some examples, the report may include an accuracy rate associated with a range of estimates 116, which may be measured against the accuracy rate of other healthcare services providers, payers, and/or estimation models 104 in an ‘apples-to-apples’ comparison. For example, the example total accuracy score 312 for the estimate associated with example account number 123 may be included in a calculation of the accuracy rate for a plurality of matched estimates 116. This accuracy rate may be compared against the accuracy rate of other estimation models 104 or against the accuracy rates in association with certain payers, healthcare providers, etc. In some examples, the information included in the report can enable an identification of issues associated with the factors or variables that impact estimate 116 accuracy (i.e., elements 202). Accordingly, the identified issues may be addressed and estimate 116 accuracy can be improved. The method 400 ends at OPERATION 498.

FIG. 5 is a block diagram illustrating physical components of an example computing device with which aspects may be practiced. The computing device 500 may include at least one processing unit 502 and a system memory 504. The system memory 504 may comprise, but is not limited to, volatile (e.g. random access memory (RAM)), non-volatile (e.g. read-only memory (ROM)), flash memory, or any combination thereof. System memory 504 may include operating system 506, one or more program instructions 508, and may include sufficient computer-executable instructions for the estimate scoring engine 120, which when executed, perform functionalities as described herein. Operating system 506, for example, may be suitable for controlling the operation of computing device 500. Furthermore, aspects may be practiced in conjunction with a graphics library, other operating systems, or any other application program and is not limited to any particular application or system. This basic configuration is illustrated by those components within a dashed line 510. Computing device 500 may also include one or more input device(s) 512 (keyboard, mouse, pen, touch input device, etc.) and one or more output device(s) 514 (e.g., display, speakers, a printer, etc.).

The computing device 500 may also include additional data storage devices (removable or non-removable) such as, for example, magnetic disks, optical disks, or tape. Such additional storage is illustrated by a removable storage 516 and a non-removable storage 518. Computing device 500 may also contain a communication connection 520 that may allow computing device 500 to communicate with other computing devices 522, such as over a network in a distributed computing environment, for example, an intranet or the Internet. Communication connection 520 is one example of a communication medium, via which computer-readable transmission media (i.e., signals) may be propagated.

Programming modules may include routines, programs, components, data structures, and other types of structures that may perform particular tasks or that may implement particular abstract data types. Moreover, aspects may be practiced with other computer system configurations, including hand-held devices, multiprocessor systems, microprocessor-based or programmable user electronics, minicomputers, mainframe computers, and the like. Aspects may also be practiced in distributed computing environments where tasks are performed by remote processing devices that are linked through a communications network. In a distributed computing environment, programming modules may be located in both local and remote memory storage devices.

Furthermore, aspects may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (So( )). Aspects may also be practiced using other technologies capable of performing logical operations such as, for example, AND, OR, and NOT, including, but not limited to, mechanical, optical, fluidic, and quantum technologies. In addition, aspects may be practiced within a general purpose computer or in any other circuits or systems.

Aspects may be implemented as a computer process (method), a computing system, or as an article of manufacture, such as a computer program product or computer-readable storage medium. The computer program product may be a computer storage medium readable by a computer system and encoding a computer program of instructions for executing a computer process. Accordingly, hardware or software (including firmware, resident software, micro-code, etc.) may provide aspects discussed herein. Aspects may take the form of a computer program product on a computer-usable or computer-readable storage medium having computer-usable or computer-readable program code embodied in the medium for use by, or in connection with, an instruction execution system.

Although aspects have been described as being associated with data stored in memory and other storage mediums, data can also be stored on or read from other types of computer-readable media, such as secondary storage devices, like hard disks, floppy disks, or a CD-ROM, or other forms of RAM or ROM. The term computer-readable storage medium refers only to devices and articles of manufacture that store data or computer-executable instructions readable by a computing device. The term computer-readable storage media do not include computer-readable transmission media.

Aspects of the present invention may be used in various distributed computing environments where tasks are performed by remote processing devices that are linked through a communications network.

Aspects of the invention may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 500 or any other computing devices 522, in combination with computing device 500, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described aspects.

The description and illustration of one or more aspects provided in this application are intended to provide a thorough and complete disclosure the full scope of the subject matter to those skilled in the art and are not intended to limit or restrict the scope of the invention as claimed in any way. The aspects, examples, and details provided in this application are considered sufficient to convey possession and enable those skilled in the art to practice the best mode of the claimed invention. Descriptions of structures, resources, operations, and acts considered well-known to those skilled in the art may be brief or omitted to avoid obscuring lesser known or unique aspects of the subject matter of this application. The claimed invention should not be construed as being limited to any embodiment, aspects, example, or detail provided in this application unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently. Having been provided with the description and illustration of the present application, one skilled in the art may envision variations, modifications, and alternate embodiments falling within the spirit of the broader aspects of the general inventive concept provided in this application that do not depart from the broader scope of the present disclosure.

1.-20. (canceled)

21. A system for providing quantitative estimate accuracy scoring of healthcare service cost estimation models, comprising:
at least one processing device; and
at least one computer readable data storage device storing instructions that, when executed by the at least one processing device, cause the at least one processing device to:
receive, from a service provider system, a first plurality of estimates that are generated by a first estimation model associated with the service provider system, wherein a first estimate of the first plurality of estimates corresponds to an estimated out-of-pocket cost for a first service rendered by the service provider system, and wherein the first estimate comprises a plurality of elements;
store, in a first data store, the first plurality of estimates;
receive, from a first payer system of a plurality of payer systems, a plurality of remittances, wherein a first remittance of the plurality of remittances corresponds to the first estimate, and wherein the first remittance comprises an actual out-of-pocket cost for the first service rendered by the service provider system;
store, in a second data store, the plurality of remittances;
generate a total accuracy score for the first estimate by:
matching the first estimate with the first remittance,
determining an accuracy score for each element of the plurality of elements, wherein each of the plurality of elements is associated with at least one factor that impacts accuracy of an estimate, and
based on the accuracy score for each of the plurality of elements, determine a total accuracy score for the first estimate;
generate and transmit, to a display device, display instructions that, when executed, cause a user interface element to be displayed by the display device, the user interface element comprising:
a payer breakout user interface component, wherein the payer breakout user interface component comprises a plurality of blocks such that a first block of the plurality of blocks associated with the first payer system comprises (i) a color determined based at least in part on the total accuracy score of the first estimate and (ii) a size determined based at least in part on a number of estimates associated with the first payer system in relation to remaining payer systems of the plurality of payer systems, wherein the payer breakout user interface component is configured to automatically update the plurality of blocks based at least in part on a change in the total accuracy score of the first estimate, and
one or more filtering options, wherein a first filtering option enables a user to select a date range;
in response to receiving an indication of a user selection of the first block, generate and transmit, to the display device, updated display instructions that, when executed, cause a first updated user interface element to be displayed by the display device, the first updated user interface element comprising: information associated with the first payer system, and at least one of a change in the size or the color of the first block;
in response to receiving an indication of a user selection of a first date range, cause a second updated user interface element to be displayed, the second updated user interface element comprising:
a subset of the plurality of blocks, wherein a second block of the plurality of blocks associated with the first payer system comprises a number of estimates associated with the first payer system that are generated on dates within the first date range; and
receive, from the service provider system, a second plurality of estimates that are generated by a second estimation model associated with the service provider system, wherein a second estimate of the second plurality of estimates comprises the plurality of elements of the first estimate, and wherein the second estimation model utilizes a different weighting than the first estimation model for at least one element of the plurality of elements, wherein the different weighting is determined based on the total accuracy score of the first estimate.

22. The system of claim 21, wherein the system is further configured to:
based on a total accuracy score for each of the first plurality of estimates, determine an accuracy rate for the first plurality of estimates.

23. The system of claim 22, wherein the system is further configured to:
compare the accuracy rate for the first plurality of estimates against an estimation accuracy goal value to determine whether the accuracy rate meets the estimation accuracy goal value; and
include a visual indication component of the accuracy rate for the first plurality of estimates in comparison with the estimation accuracy goal value in the user interface element.

24. The system of claim 21, wherein the user interface element comprises instructions for rendering a user interface on a remote user device.

25. The system of claim 21, wherein the plurality of elements comprises at least one of: chargemaster amount, contractual adjustment amount, patient benefit amount, or patient responsibility amount.

26. The system of claim 21, wherein to determine the accuracy score for each of the plurality of elements, the instructions further cause the at least one processing device to:
match a charge description master value associated with the first estimate against an actual charge description master value associated with the first remittance;
apply scoring criteria; and
determine a score for a charge description master value element based on the scoring criteria.

27. The system of claim 21, wherein to determine the accuracy score for each of the plurality of elements, the instructions further cause the at least one processing device to:
match a contractual adjustment value associated with the first estimate against an actual contractual adjustment value associated with the first remittance;
apply scoring criteria; and
determine a score for a contractual adjustment value element based on the scoring criteria.

28. The system of claim 21, wherein to determine the accuracy score for each of the plurality of elements, the instructions further cause the at least one processing device to:
match a copayment value associated with the first estimate against an actual copayment value associated with the first remittance;
apply scoring criteria; and
determine a score for a copayment value element based on the scoring criteria.

29. The system of claim 21, wherein to determine the accuracy score for each of the plurality of elements, the instructions further cause the at least one processing device to:
match a deductible value associated with the first estimate against an actual deductible value associated with the first remittance;
apply scoring criteria; and
determine a score for a deductible value element based on the scoring criteria.

30. The system of claim 21, wherein to determine the accuracy score for each of the plurality of elements, the instructions further cause the at least one processing device to:
match a coinsurance value associated with the first estimate against an actual coinsurance value associated with the first remittance;
apply scoring criteria; and
determine a score for a coinsurance value element based on the scoring criteria.

31. The system of claim 21, wherein to determine the accuracy score for each of the plurality of elements associated with the estimate, the instructions further cause the at least one processing device to:
match a patient responsibility value associated with the first estimate against an actual patient responsibility value associated with the first remittance;
apply scoring criteria; and
determine a score for a patient responsibility value element based on the scoring criteria.

32. A method for providing quantitative estimate accuracy scoring of healthcare service cost estimation models, comprising:
receiving, from a service provider system, a first plurality of estimates that are generated by a first estimation model associated with the service provider system, wherein a first estimate of the first plurality of estimates corresponds to an estimated out-of-pocket cost for a first service rendered by the service provider system, and wherein the first estimate comprises a plurality of elements;
storing, in a first data store, the first plurality of estimates;
receiving, from a first payer system of a plurality of payer systems, a plurality of remittances, wherein a first remittance of the plurality of remittances corresponds to the first estimate, and wherein the first remittance comprises an actual out-of-pocket cost for the first service rendered by the service provider system;
storing, in a second data store, the plurality of remittances;
generating a total accuracy score for the first estimate by:
matching the first estimate with the first remittance,
determining an accuracy score for each element of the plurality of elements, wherein each of the plurality of elements is associated with at least one factor that impacts accuracy of an estimate, and
based on the accuracy score for each of the plurality of elements, determining, a total accuracy score for the first estimate;
generating and transmitting, to a display device, display instructions that when executed, cause a user interface element to be displayed by the display device, the user interface element comprising:
a payer breakout user interface component, wherein the payer breakout user interface component comprises a plurality of blocks such that a first block of the plurality of blocks associated with the first payer system comprises (i) a color determined based at least in part on the total accuracy score of the first estimate and (ii) a size determined based at least in part on a number of estimates associated with the first payer system in relation to remaining payer systems of the plurality of payer systems, wherein the payer breakout user interface component is configured to automatically update the plurality of blocks based at least in part on a change in the total accuracy score of the first estimate, and
one or more filtering options, wherein a first filtering option enables a user to select a date range;
in response to receiving an indication of a user selection of the first block, generating and transmitting, to the display device, updated display instructions that, when executed, cause a first updated user interface element to be displayed by the display device, the first updated user interface element comprising:
information associated with the first payer system, and
at least one of a change in the size or the color of the first block;
in response to receiving an indication of a user selection of a first date range, cause a second updated user interface element to be displayed, the second updated user interface element comprising:
a subset of the plurality of blocks, wherein a second block of the plurality of blocks associated with the first payer system comprises a number of estimates associated with the first payer system that are generated on dates within the first date range; and
receiving, from the service provider system, a second plurality of estimates that are generated by a second estimation model associated with the service provider system, wherein a second estimate of the second plurality of estimates comprises the plurality of elements of the first estimate, and wherein the second estimation model utilizes a different weighting than the first estimation model for at least one element of the plurality of elements, wherein the different weighting is determined based on the total accuracy score of the first estimate.

33. The method of claim 32, further comprising:
based on a total accuracy score for each of the first plurality of estimates, determining an accuracy rate for the first plurality of estimates;
comparing the accuracy rate for the first plurality of estimates against an estimation accuracy goal value to determine whether the accuracy rate meets the estimation accuracy goal value; and
including a visual indication component of the accuracy rate for the first plurality of estimates in comparison with the estimation accuracy goal value in the user interface element.

34. The method of claim 32, wherein the user interface element comprises instructions for rendering a user interface on a remote user device.

35. The method of claim 32, wherein the plurality of elements comprises at least one of: chargemaster amount, contractual adjustment amount, patient benefit amount, or patient responsibility amount.

36. The method of claim 32, wherein determining the accuracy score for each of the plurality of elements associated with the estimate comprises:
matching a charge description master value associated with the first estimate against an actual charge description master value associated with the first remittance;
matching a contractual adjustment value associated with the first estimate against an actual charge description master value associated with the first remittance;
matching a copayment value associated with the first estimate against an actual copayment value associated with the first remittance;
matching a deductible value associated with the first estimate against an actual copayment value associated with the first remittance;
matching a coinsurance value associated with the first estimate against an actual coinsurance value associated with the first remittance;
matching a patient responsibility value associated with the first estimate against an actual patient responsibility value associated with the first remittance;
applying scoring criteria to the matched values; and
determining, based on the scoring criteria:
a charge description master value element score;
a contractual adjustment value element score;
a copayment value element score;
a deductible value element score;
a coinsurance value element score; and
a patient responsibility value element score.

37. A non-transitory computer-readable storage device comprising computer readable instructions, which when executed by a processing unit, are configured to:
receive, from a service provider system, a first plurality of estimates that are generated by a first estimation model associated with the service provider system, wherein a first estimate of the first plurality of estimates corresponds to an estimated out-of-pocket cost for a first service rendered by the service provider system, and wherein the first estimate comprises a plurality of elements;
store, in a first data store, the first plurality of estimates;
receive, from a first payer system of a plurality of payer systems, a plurality of remittances, wherein a first remittance of the plurality of remittances corresponds to the first estimate, and wherein the first remittance comprises an actual out-of-pocket cost for the first service rendered by the service provider system;
store, in a second data store, the plurality of remittances;
generate a total accuracy score for the first estimate by:
matching the first estimate with the first remittance,
determining an accuracy score for each of the plurality of elements, wherein each of the plurality of elements is associated with at least one factor that impacts accuracy of an estimate, and
based on the accuracy score for each of the plurality of elements, determine a total accuracy score for the first estimate;
generate and transmit, to a display device, display instructions that, when executed, cause a user interface element to be displayed by the display device, the user interface element comprising:
a payer breakout user interface component, wherein the payer breakout user interface component comprises a plurality of blocks such that a first block of the plurality of blocks associated with the first payer system comprises (i) a color determined based at least in part on the total accuracy score of the first estimate and (ii) a size determined based at least in part on a number of estimates associated with the first payer system in relation to remaining payer systems of the plurality of payer systems, wherein the payer breakout user interface component is configured to automatically update the plurality of blocks based at least in part on a change in the total accuracy score of the first estimate, and
one or more filtering options, wherein a first filtering option enables a user to select a date range;
in response to receiving an indication of a user selection of the first block, generate and transmit, to the display device, updated display instructions that, when executed, cause a first updated user interface element to be displayed by the display device, the first updated user interface element comprising:
information associated with the first payer system, and
at least one of a change in the size or the color of the first block;
in response to receiving an indication of a user selection of a first date range, cause a second updated user interface element to be displayed, the second updated user interface element comprising:
a subset of the plurality of blocks, wherein a second block of the plurality of blocks associated with the first payer system comprises a number of estimates associated with the first payer system that are generated on dates within the first date range; and
receive, from the service provider system, a second plurality of estimates that are generated by a second estimation model associated with the service provider system, wherein a second estimate of the second plurality of estimates comprises the plurality of elements of the first estimate, and wherein the second estimation model utilizes a different weighting than the first estimation model for at least one element of the plurality of elements, wherein the different weighting is determined based on the total accuracy score of the first estimate.

38. The non-transitory computer-readable storage device of claim 37, wherein the instructions, when executed by the processing unit, are further configured to:
based on a total accuracy score for each of the first plurality of estimates, determine an accuracy rate for the first plurality of estimates;
compare the accuracy rate for the first plurality of estimates against an estimation accuracy goal value to determine whether the accuracy rate meets the estimation accuracy goal value; and
include a visual indication component of the accuracy rate for the first plurality of estimates in comparison with the estimation accuracy goal value in the user interface element.

39. The non-transitory computer-readable storage device of claim 38, wherein the instructions, when executed by the processing unit, are further configured to:
determine a number of estimates included in the first plurality of estimates that are associated with the first payer system;
determine an amount by which a total of patient responsibility values included in the first plurality of estimates differ from a total of actual patient responsibility values; and
include, in the user interface element, a visual indication of:
the number of estimates included in the first plurality of estimates that are associated with the first payer system;
the amount by which the total of patient responsibility values included in the first plurality of estimates differ from the total of actual patient responsibility values;
whether the total of patient responsibility values included in the first plurality of estimates is greater than or less than the total of actual patient responsibility values; and
a degree by which the total of patient responsibility values included in the first plurality of estimates is greater than or less than the total of actual patient responsibility values.

40. The non-transitory computer-readable storage device of claim 37, wherein the instructions, when executed by the processing unit, are further configured to:
match a charge description master value associated with the first estimate against an actual charge description master value associated with the first remittance;
match a contractual adjustment value associated with the first estimate against an actual charge description master value associated with the first remittance;
match a copayment value associated with the first estimate against an actual copayment value associated with the first remittance;
match a deductible value associated with the first estimate against an actual copayment value associated with the first remittance;
match a coinsurance value associated with the first estimate against an actual coinsurance value associated with the first remittance;
match a patient responsibility value associated with the first estimate against an actual patient responsibility value associated with the first remittance;
apply scoring criteria to the matched values; and
determine, based on the scoring criteria:
a charge description master value element score;
a contractual adjustment value element score;
a copayment value element score;
a deductible value element score;
a coinsurance value element score; and
a patient responsibility value element score.
